(19) United States
(12) Patent Application Publication
(10) Pub. No.: US 2012/0282200 A1
(43) Pub. Date: Nov. 8, 2012

(54) UV ABSORBING DENTRITIC POLYETHER PREPARED BY POLYMERIZATION OF OXETANES

(76) Inventor: Alexander Schlifke-Poschalko, Basel (CH)

(21) Appl. No.: 13/505,832

(22) PCT Filed: Dec. 8, 2010
(86) PCT No.: PCT/EP2010/069132

§ 371 Date: Jul. 24, 2012

(30) Foreign Application Priority Data
Dec. 9, 2009 (EP) 09178478.5

(51) Int. Cl. C07C 229/60; A61Q 17/04; A61K 8/45

(52) U.S. Cl. 424/60; 560/50

(57) ABSTRACT

The present invention relates to a polymer comprising a hyperbranched dendritic polyether backbone covalently bonded via an oxygen bridge to at least one UV absorbing chromophore characterized in that the hyperbranched dendritic polyether backbone is obtainable by a ring-opening polymerization of at least one oxetane. Such polymers are particularly useful as UV-filter substances for example in compositions for the protection of the human skin and/or hair against harmful effects of sunlight.

The present invention relates to a polymer comprising a hyperbranched dendritic polyether backbone covalently bonded via an oxygen bridge to at least one UV absorbing chromophore characterized in that the hyperbranched dendritic polyether backbone is obtainable by a ring-opening polymerization of at least one oxetane. Such polymers are particularly useful as UV-filter substances for example in compositions for the protection of the human skin and/or hair against harmful effects of sunlight.

Many UV-filter substances have been developed in the past protecting against the harmful effect of UV-A (320-400 nm) and/or UV-B (290-320 nm) wavelength and even shorter wavelengths (UV-C). These chemicals are usually incorporated either alone or in combination with each other in cosmetic or pharmaceutical preparations which are widely known and used.

There are many requirements on a UV-filter substance such as having excellent photostability, toxicological and dermatological acceptability, excellent heat stability, very good solubility in cosmetic solvents or water, processability into cosmetic formulations, compatibility with cosmetic bases, pH stability in the range of 4 to 9, compatibility with other ingredients of cosmetic formulations and with the packaging materials, no staining of textiles, it should be free of color and of neutral or pleasant odor, and it should be free of tackiness and have a low volatility.

Polymeric UV-filter substances have received increasing attention during the last decade as government authorities place more and more stringent requirements on sunscreens in view of transdermal penetration in order to avoid any systemic exposure. But even though many polymeric UV-filters have been disclosed in the literature, this class still suffers from many disadvantages. At high chromophore loading the polymeric UV-filter substances are often highly viscous, honey-like products which are difficult to process industrially. Furthermore, they often show a lack of photostability as well as solubility, are laborious to prepare and to purify and, incorporated into a topical composition and applied to the skin lead to an unpleasant skin feel. At low chromophore loadings, the polymeric UV-filter substances often do not provide a sufficient sun protection factor (SPF).

Therefore, there is an ongoing need for novel polymeric UV-filter substances which meet the above mentioned requirement and which are in particular industrial processible even at a high chromophore loading.

WO2005/092282 relates inter alia to a conjugate comprising a hyperbranched polymer covalently bonded to at least three UV absorbing chromophores having an UV absorption maximum λₘₐₓ≧270 nm, wherein the hyperbranched polymer is obtainable by polycondensation or polyaddition of one ore more building blocks AB₂. Preferably, the building block AB₂ is glycidol. The glycidol based UV-filter substances disclosed in WO2005/092282, however, are highly viscous oils which renders their handling very difficult. Furthermore, they exhibit a limited solubility in cosmetic oils.

Hyperbranched dendritic polymers are known from the prior art and encompasses dendrimers and hyperbranched polymers. The most characteristic feature of dendritic polymers in contrast to linear polymers or ordinarily and randomly branched polymers is the absence of entanglements and the low viscosity in bulk.

Dendritic polymers are based on for instance ABₓ monomers and are highly branched macromolecules with a multitude of end-groups. Dendrimers are monodisperse, exact structures where all branch points are utilized. Due to their well defined structure dendrimers are tedious and often expensive to synthesize. Hyperbranched polymers are polydisperse and some of the ABₓ monomers are incorporated in a linear fashion, resulting in a less well defined architecture. Hyperbranched polymers are less complicated to synthesize and, therefore, less expensive which makes them more attractive than dendrimers for large-scale applications.

Surprisingly, it has been found that polymers comprising a hyperbranched dendritic polyether backbone covalently bonded via an oxygen bridge to at least one UV absorbing chromophore characterized in that the hyperbranched dendritic polyether backbone is obtainable by a ring-opening polymerization of at least one oxetane as AB₂ monomer overcome the disadvantages of the prior art outlined above. In particular the inventive polymers show a good solubility in cosmetic solvents as well as an outstanding skin compatibility and stability (light, heat, moisture). Furthermore, at high chromophore loading, the polymers according to the invention are solid and can thus easily be handled even in industrial scale. Compositions comprising a polymer according to the invention furthermore exhibit excellent sensorial properties e.g. in regard of skin feel.

Thus, the invention relates to a polymer comprising a hyperbranched dendritic polyether backbone covalently bonded via an oxygen bridge to at least one UV absorbing chromophore characterized in that the hyperbranched dendritic polyether backbone is obtainable by a ring-opening polymerization of at least one oxetane.

The oxetane is preferably a compound of the general formula (I)

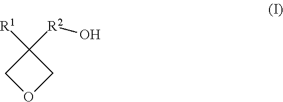

wherein
R¹ represents H, a C₁ to C₄ alkyl group, an aryl group or an arylalkyl group and
R² represents a C₁ to C₄ alkylene group.

Examples of alkyl groups which may be represented by R¹ are methyl, ethyl, propyl, isopropyl, butyl, isobutyl, sec.-butyl and t-butylgroups of which the preferred groups are methyl and ethyl, especially the methyl groups. Examples of alkylene groups which may be represented by R² include the methylene, ethylene, trimethylene, propylene, tetramethylene, ethylethylene and methyltrimethylene groups of which the methylene and ethylene groups are preferred, the methylene group being the most preferred. In all embodiments of the invention preferably R¹ is methyl or ethyl and R² is methyl. The most preferred oxetane in all embodiments of the invention is 3-methyl-3-hydroxymethyl oxetane.

The hyperbranched dendritic polyether backbone is obtainable by a one step polymerization using oxetane as AB₂-type monomer i.e. building block. The polymerization reaction may be initiated by the oxetane itself or by a polyol starter unit. In particular, the hyperbranched dendritic polyether backbone is obtainable by a ring-opening polymerization of oxetane with the definitions and preferences as given above. In all embodiment of the invention preferably a polyol starter unit is used in the preparation of the hyperbranched dendritic polyether backbone. The hyperbranched dendritic polyether backbone according to the invention has terminal hydroxyl groups which are suitable for the attachment of the at least one UV absorbing chromophore.

Examples of polyol starter units include glycol, 1,4-cyclohexanedimethanol, hydroquinone bis(2-hydroxyethyl) ether, 2,2′-thiodiethanol, N-methyldiethanolamine, N-ethyldiethanolamine, 1,4-butanediol, 1,5-pentanediol, 1,6-hexanediol, 1,7-heptanediol, 1,8-octanediol, 1,9-nonanediol, 2-butene-1,4-diol, diethylene glycol, triethylene glycol, hexaethylene glycol, dipropylene glycol, polyethylene glycol, polypropylene glycol, tripentaerythritol, 1,2,6-hexanetriol, glycidol, 1,3,5-tris(2-hydroxyethyl)cyanuric acid, 1,3-bis[tris(hydroxymethyl)methylamino]propane, BIS-TRIS [2,2-bis(hydroxyethyl)-(iminotris)-(hydroxymethyl)-methane], N,N,N′,N′-tetrakis(2-hydroxyethyl) ethylenediamine, triethanolamine, diglycerol, glucose, fructose, sucrose, galactose, lactose, maltose, mannitol, dulcitol, threitol, sorbitol. In all embodiments of the invention the polyol starter unit is preferably selected from pentaerythritol, dipentaerythritol and/or trimethylolpropane, most preferably the polyol starter unit is trimethylolpropane.

The term ring-opening polymerization refers to a form of addition polymerization, in which the terminal end of a polymer acts as a reactive center where further cyclic monomeric building blocks, i.e. the oxetane join to form a larger polymer chain through ionic propagation. When the reactive center of the propagating chain is a carbocation the polymerization is called cationic ring-opening polymerization and when the active center is a carbanion the reaction is an anionic ring-opening polymerization. The ring-opening polymerization is performed using an effective amount of at least one catalyst, such as e.g. a base or an acid. Suitable catalysts are e.g. Lewis acids such as AlCl₃, FeCl₃, SnCl₄, and BF₃ and/or Brønsted acids such as naphthalene sulphonic acid, para-toluene sulphonic acid, methane sulphonic acid, trifluoromethane sulphonic acid, trifluoroacetic acid, sulphuric acid and/or phosphoric acid, and/or onium salts, alcoholates such as e.g. potassium tert.-butylate or potassium methylate without being limited thereto. Further catalysts are alkali metals such as potassium or sodium, and alkali metal hydrides such as potassium hydride and sodium hydrid. In all embodiments of the invention preferred catalysts are potassium hydride and/or potassium methylate.

An exemplary process for the preparation of a hyperbranched dendritic polyether backbone using 3-methyl-3-hydroxymethyl oxetane as starter as well as building block is illustrated below. In basic medium the primary hydroxyl groups of 3-methyl-3-hydroxymethyl oxetane are deprotonated thereby initiating an anionic ring-opening multibranching polymerization (ROMBP).

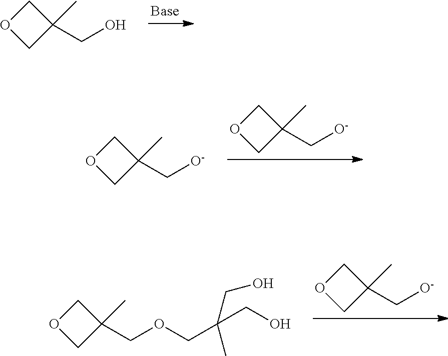

An exemplary process for the preparation of the hyperbranched dendritic polyether backbone using trimethylol propane as polyol starter unit and 3-methyl-3-hydroxymethyl oxetane as building block is depicted below:

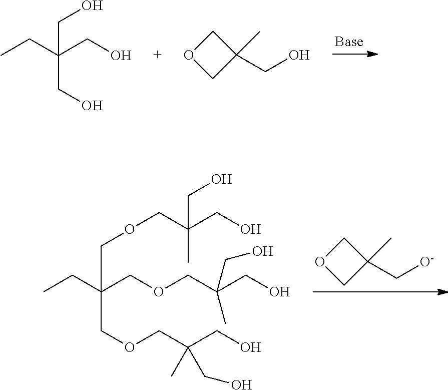

Further information on the preparation of hyperbranched dendritic oxetane based polyether backbones suitable for the subsequent coupling of UV-filter substances are e.g. disclosed in WO0056802 or J. Polym. Sci. Part. A.: Polym. Chem., 36, 1685 (1998) which are enclosed herein by reference.

In all embodiments of the invention the hyperbranched dendritic polyether backbone comprises at least one oxetane building block with the preferences and definitions as given above per molecule. Preferably the hyperbranched dendritic polyether backbone comprises an average number of 2 to 600, more preferably of 2 to 250, most preferably of 3 to 100, in particular of 4 to 25 oxetane building blocks per molecule. In a particular preferred embodiment the hyperbranched dendritic polyether backbone is obtainable by a ring-opening polymerization of only oxetane with the preferences and definitions as given above as building block and wherein either the oxetane itself acts as starter unit or a polyol starter unit is used. Preferably, a polyol starter unit with the definitions and preferences as given above is used.

Thus, in a specific embodiment the invention relates to a hyperbranched dendritic polyether backbone covalently bonded via an oxygen bridge to at least one UV absorbing chromophore characterized in that the hyperbranched dendritic polyether backbone is obtainable by a ring-opening polymerization of oxetane, optionally using a polyol starter unit. In a particular preferred embodiment the oxetane is 3-methyl-3-hydroxymethyl oxetane and/or 3-ethyl-3-hydroxymethyl oxetane and the polyol starter unit is selected from pentaerythritol, dipentaerythritol and/or trimethylolpropane, most preferably the oxetane is 3-methyl-3-hydroxymethyl and the polyol starter unit is trimethylolpropane.

The hyperbranched dendritic polyether backbone is covalently bound to at least one, preferably to at least two, more preferably to at least three, most preferably to at least four, in particular to at least five UV absorbing chromophores. If desirable, all terminal hydroxyl groups of the hyperbranched dendritic polyether backbone can be functionalized with a UV absorbing chromophore. When more than one UV absorbing chromophore is present in the polymer according to the invention, the UV absorbing chromophores may be of a different or of the same type. Preferably, all UV absorbing chromophores are identical.

The UV absorbing chromophores covalently bound via an oxygen bridge to the hyperbranched dendritic polyether backbone comprise all groups which absorb light in the range of wavelengths 400 nm to 320 nm (UV-A) and 320 nm to 290 nm (UV-B) or of even shorter wavelengths (UV-C) and which are or can be used as chemical UV-filter substances. Preferably, the UV absorbing chromophores are derived from chromophores which are known UV-filter substances. Most preferably, the UV absorbing chromophores are derived from commercially available UV-filter substances as e.g. listed in the CTFA Cosmetic ingredient Handbook or “The Encyclopedia of Ultraviolet Filters” (ISBN: 978-1-932633-25-2) by Nadim A. Shaath.

Suitable UV absorbing chromophores encompass derivatives of acrylates such as e.g. 2-cyano-3,3-diphenylacrylate, of p-aminobenzoates such as e.g. p-dimethylaminobenzoate, of camphor such as e.g. 4-methyl benzylidene camphor, of cinnamates such as e.g. 4-methoxycinnamate, of benzophenones such as e.g. the 2,4-dihydroxybenzophenone or 4-amino-2 hydroxybenzophenone, of benzalmalonates, of 2-(4-ethoxy anilinomethylene)propanoates, of imidazole, of salicylates, of triazones, of triazols such as e.g. 2-hydroxyphenyl-benzotriazols, of dibenzoylmethanes such as e.g. of the t-butyl-dibenzoylmethane, of phenyl-benzimidazoles, of anthranilates, of phenyl-benzoxazoles, of 1,4-dihydropyranes and of others representing state of the art and known to those skilled in the art to be highly active.

In all embodiments of the invention, preferred groups of UV absorbing chromophores are groups derived from p-aminobenzoic acid such as e.g. p-dimethylaminobenzoic acid, amino substituted hydroxybenzophenones such as e.g. 2-[4-diethylaminobenzoyl]benzoic acid, cinnamic acid such as e.g. 4-methoxycinnamic acid, benzalmalonate such as e.g. [(4-hydroxyphenyl)methylene] propanedioate, in particular groups derived from p-aminobenzoic acid such as p-dimethylaminobenzoic acid.

The at least one UV absorbing chromophore is covalently bound via an oxygen bridge to the hyperbranched dendritic polyether backbone. The oxygen atom of the oxygen bridge may either be derived from a hydroxyl group of the UV absorbing chromophore or from the terminal hydroxyl groups of the hyperbranched dendritic polyether backbone. The linkage can be in the form of an ether-, an ester-, an urethane-, an amide-, a phenol ether- or a carbonate-group without being limited thereto. Preferably, the linkage is in the form of an ether-or an ester-group, most preferably the linkage is an ester group.

If the oxygen atom of the oxygen bridge is derived from the terminal hydroxyl groups of the hyperbranched dendritic polyether backbone, the polymers according to the present invention may be prepared by reacting the terminal hydroxyl groups of the hyperbranched dendritic polyether backbone with an UV absorbing chromophore as defined above having a functional group capable of reacting with the hydroxyl groups of the hyperbranched dendritic polyether backbone. Suitable functional groups include carboxyl-, ester-, acylhalogenide-, halogenide, anhydride, epoxide or isocyanate groups without being limited thereto.

The attachment can be performed by known method to a person skilled in the art, e.g. by reacting an acid chloride of a UV absorbing chromophore with the terminal hydroxyl groups of the hyperbranched dendritic polyether in the presence of a base such as pyridine. An alternative method includes the transesterification of a UV-absorbing chromophore which is an ester such as e.g. an ester of p-aminobenzoate, an ester of 2-cyano-3,3-diphenylacrylate, an ester of 2-[4-(diethylamino)-2-hydroxybenzoyl]-benzoic acid or an ester of 2-methoxycinnamate with the terminal hydroxyl groups of the hyperbranched dendritic polyether. Suitable esters are for example the methyl or the ethyl esters, in particular the methyl esters.

If the oxygen atom of the oxygen bridge is derived from a hydroxyl group of the UV-absorbing chromophore, then the terminal hydroxyl groups of the hyperbranched dendritic polyether backbone must be activated prior to the reaction. In this case the terminal hydroxyl groups of the hyperbranched dendritic polyether backbone are converted to leaving groups such as e.g. bromides, chlorides, mesylates, tosylates or triflates. The procedures to convert a hydroxyl group in a leaving group are known to one skilled in this art. For example, the terminal hydroxyl groups of the hyperbranched dendritic polyether may be reacted with mesylchloride in the presence of a tertiary amine or with SOCl₂ in order to enhance the leaving group quality of the hydroxyl groups of the hyperbranched dendritic polyether backbone (activation). Afterwards, the activated hyperbranched dendritic polyether backbone is reacted e.g. with a deprotonated hydroxyl group of a suitable UV absorbing chromophore in a nucleophilic substitution reaction thereby forming an ether bond.

In all embodiments of the invention, preferably the terminal hydroxyl groups of the hyperbranched dendritic polyether backbone are reacted with an acid chloride or an ester of a UV absorbing chromophore. Most preferably in all embodiments of the invention the UV absorbing chromophore which is reacted with the terminal hydroxyl groups of the hyperbranched dendritic polyether backbone is an ester of p-dimethylaminobenzoic acid such as in particular methyl p-dimethylaminobenzoate, ethyl p-dimethylaminobenzoate, methyl p-diethylaminobenzoate or ethyl p-diethylaminobenzoate most in particular methyl p-dimethylaminobenzoate or ethyl p-dimethylaminobenzoate.

If not all terminal hydroxyl groups of the hyperbranched polyether backbone are reacted with a UV absorbing chromophore the residual hydroxyl groups may remain present in the polymer according to the invention. If desired, the residual hydroxyl groups can be reacted with suitable capping agents. Suitable capping agents include anhydrides or acid chlorides or acid esters of C₁ to C₂₀ linear or branched alkanoic acids such as such as e.g. acetanhydride, acetylchloride, 2-ethyl hexanoic acid (m)ethyl ester or 2-ethyl hexanoic acid chloride. The skilled person is aware of further suitable capping agents which can be used to introduce the corresponding capping groups. In all embodiments of the invention preferably the residual hydroxyl groups of the hyperbranched dendritic polyether according to the invention are capped, preferably with acetanhydride, acetyl chloride and/or 2-ethyl hexanoic acid chloride or ester, most preferably with 2-ethyl hexanoic acid chloride or ester.

In all embodiments of the invention preferably 30-100%, most preferably 70-90% of the terminal hydroxyl groups of the hyperbranched dendritic polyether backbone are linked to a UV absorbing chromophore and the residual terminal hydroxyl groups are linked to a capping group, in particular to a 2-ethyl hexanoyl-group.

Thus, in a particular embodiment, the invention relates to a polymer consisting of a hyperbranched dendritic polyether backbone covalently bonded via an oxygen bridge to at least one UV absorbing chromophore characterized in that the hyperbranched dendritic polyether backbone is obtainable by a ring-opening polymerization of oxetane, optionally and preferably in the presence of a polyol starter unit, in particular trimethylolpropane and wherein 30-100%, most preferably 70-90% of the terminal hydroxyl groups of the hyperbranched dendritic polyether backbone are linked to a UV absorbing chromophore and the residual terminal hydroxyl groups are linked to a capping group, in particular to a 2-ethyl hexanoyl-group. In a particular preferred embodiment, the oxetane is 3-methyl-3-hydroxymethyl oxetane, the polyol starter unit is trimethylolpropane, 70-90% of the terminal hydroxyl groups of the hyperbranched dendritic polyether backbone are linked to the same UV absorbing chromophore and the residual hydroxyl groups are linked to a 2-ethyl hexanoyl-group.

Preferably in all embodiments of the invention the polymer according to the invention has a number average molecular weight Mₙ within the range of from 500 to 50,000 g mol⁻¹, more preferably 750 to 25,000 g mol⁻¹, most preferably 1,000 to 10,000 g mol⁻¹. The number average molecular weight Mn can be determined by Gel Permeation Chromatography (GPC) as described in the DIN 55672-1 using e.g. polystyrene standards.

The photostability of the polymer according to the present invention may be measured according to G. Berset et al. International Journal of Cosmetic Science 1996, 18(3), 167-177.

The polymers according to the present invention are particularly useful as UV filters substances, i.e. for protecting ultraviolet-sensitive organic materials, in particular the skin and hair of humans and animals from the harmful effects of UV radiation. The polymers according to the invention are not only suitable for “immediate protection from acute sun damage” such as sun burn (sun erythema), but also protect against damages through sunlight-induced oxidative stress and/or immune suppression and/or their consequences, i.e. photoaging. Furthermore, the polymers according to the present invention are also suitable to protect natural or artificial hair color.

The polymers in accordance with the invention are colorless or light yellowish, liquid, crystalline or semi-liquid substances. They are distinguished by high photostability, good solubility in organic solvents, especially cosmetic solvents, and a short and economical synthetic route.

Due to their good lipophilicity the polymers of the invention can be incorporated well into oil- and/or fat-containing topical compositions.

With respect to the lipophilicity the polymers of the invention in accordance with the invention are superior to comparable glycidol based UV-filter substances referred to earlier as the polymers of the invention fulfill the criteria which are required in the present instance, namely a high solubility in cosmetic solvents such as e.g. C12-15 alkyl benzoate (e.g., FINSOLV TN [Finetex Inc.]), isopropyl myristate (e.g., DELTYL EXTRA, [Givaudan Roure Vernier, Switzerland]), PPG 15-stearyl ether (e.g., ARLAMOL E, [ICI Surfactants Everberg 1 Belgium]), cocoyl capylate/caprate (e.g., CETIOL LC, [sold by Henkel KGaA, Dusseldorf Germany]) or diisopropyl adipate (e.g., CRODAMOL DA, [Croda Universal Ltd., North Humberside, England]) which is further illustrated in the examples.

The polymers according to the invention are therefore suitable for the incorporation as UV-filter substances in topical compositions such as in cosmetic, pharmaceutical and veterinary medical preparations. The polymers can be used both in dissolved form and in the micronized state.

Thus, the present invention also relates to compositions, preferably to topical compositions comprising a polymer according to the invention and a cosmetically acceptable carrier.

The amount of the polymer according to the invention in the compositions according to the invention is not critical. Preferably an amount of at least 0.01 wt.-% is used. More preferably an amount of 0.5 to 20 wt.-%, in particular 1 to 10 wt.-% such as e.g. from about 2 to 5 wt.-% based on the total weight of the composition are incorporated.

The term “topical composition” as used herein refers in particular to a cosmetic composition that can be topically applied to mammalian keratinous tissue, particularly human skin and hair.

The term “cosmetic preparation” or “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Preparations”, Verlag für chemische Industrie (ed. H. Ziolkowsky), 4^{th} edition, 1992.

Preferably, the topical compositions according to the present invention are in the form of a suspension or dispersion in solvents or fatty substances, or alternatively in the form of an emulsion or micro emulsion (in particular of O/W- or W/O-type), PIT-emulsion, multiple emulsion (e. g. O/W/O- or W/O/W-type), pickering emulsion, hydrogel, alcoholic gel, lipogel, one- or multiphase solution or vesicular dispersion or other usual forms, which can also be applied by pens, as masks or as sprays. If the topical composition is or comprises an emulsion it can also contain one or more anionic, nonionic, cationic or amphoteric surfactant(s).

Preferred topical compositions according to the invention are skin care preparations, decorative preparations, light protection preparations and functional preparations.

Examples of skin care preparations are, in particular, body oils, body lotions, body gels, treatment creams, skin protection ointments, shaving preparations, such as shaving foams or gels, skin powders such as baby powder, moisturizing gels, moisturizing sprays, revitalizing body sprays, cellulite gels, face and/or body moisturizers, facial and/or body cleansers, face masks, anti acne preparations and/or peeling preparations.

Examples of decorative preparations are, in particular, lipsticks, eye shadows, mascaras, dry and moist make-up formulations, rouges, powders, and/or suntan lotions.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

Topical compositions in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as a aerosol mousse, a foam or a spray foam, a spray, a stick, a plaster, a cleanser, a soap or a wipe.

In accordance with the present invention, the topical composition contains at least one polymer according to the invention, optionally in combination with further ingredients such as ingredients for skin lightening; tanning prevention; treatment of hyperpigmentation;

preventing or reducing acne, wrinkles, lines, atrophy and/or inflammation; as well as topical anesthetics; antimicrobial and/or antifungal agents; chelators and/or sequestrants; anti-cellulites agents (e.g. phytanic acid) and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person.

The cosmetically active ingredients useful herein can in some instances provide more than one benefit or operate via more than one mode of action.

Examples of cosmetically active ingredients to be used in the topical composition according to the invention comprise peptides (e.g., Matrixyl™ [pentapeptide derivative]), oligopeptides, wax-based synthetic peptides (e.g., octyl palmitate and tribehenin and sorbitan isostearate and palmitoyl-oligopeptide), iodopropyl butylcarbamate, glycerol, urea, guanidine (e.g. amino guanidine); vitamins and derivatives thereof such as vitamin C (ascorbic acid), vitamin A (e.g., retinoid derivatives such as retinyl palmitate or retinyl propionate), vitamin E (e.g., tocopherol acetate), vitamin B₃ (e.g. niacinamide) and vitamin B₅ (e.g. panthenol), vitamin B₆ and vitamin B₁₂, biotin, folic acid; anti-acne actives or medicaments (e.g.

resorcinol, salicylic acid, and the like); antioxidants (e.g. phytosterols, lipoic acid); flavonoids (e.g. isoflavones, phytoestrogens); skin soothing and healing agents such as aloe vera extract, allantoin and the like; agents suitable for aesthetic purposes such as essential oils, fragrances, skin sensates, opacifiers, aromatic compounds (e.g., clove oil, menthol, camphor, eucalyptus oil, and eugenol), desquamatory actives, hydroxy acids such as AHA acids, poly unsaturated fatty acids, radical scavengers, farnesol, antifungal actives in particular bisabolol, alkyldiols such as 1,2-pentanediol, hexanediol or 1,2-octanediol, phytol, polyols such as phytanetriol, ceramides and pseudoceramides, amino acids, protein hydrolysates, polyunsaturated fatty acids, plant extracts like kinetin, DNA or RNA and their fragmentation products, carbohydrates, conjugated fatty acids, carnitin, carnosine, biochinonen, phytofluen, phytoen, and their corresponding derivatives, co-enzyme Q10/ubiquinone), anti-oxidants [preferably (−)-epigallocatechin galate (EGCG), hydroxytyrosol, and/or olive extract without being limited thereto.

Preferred examples of cosmetically active ingredients are vitamin C (ascorbic acid) and/or its derivatives (e.g. ascorbyl phosphate such as Stay C (sodium ascorbyl monophosphate) from DSM Nutritional Products Ltd.), vitamin A and/or its derivatives (e.g., retinoid derivatives such as retinyl palmitate or retinyl propionate), vitamin E and/or its derivatives (e.g., tocopherol acetate), vitamin B₆, vitamin B₁₂, biotin, co-enzyme Q10, EGCG, hydroxytyrosol and/or olive extract.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992) without being limited thereto.

The necessary amounts of the cosmetic and dermatological adjuvants and additives can—based on the desired product—easily be chosen by a skilled person in this field and will be illustrated in the examples, without being limited hereto.

The usual cosmetic adjuvants and additives such as e.g. emulsifiers, thickeners, surface active ingredients and film formers can show synergistic effects which can be determined by the expert in the field with normal trials, or with the usual considerations regarding the formulation of cosmetic composition.

Which amount of the topical composition has to be applied, depends on the concentration of the active ingredient(s) in the product and the desired cosmetic effect(s). A typical “leave-on” composition like a skin care emulsion or light-protective preparation, for example, is usually applied in an amount of about 0.5 to about 2 mg per cm² skin. The applied amount is normally not critical, and the desired effect(s) may be achieved by using more of the composition, repeating the application of the composition and/or applying a composition which contains more of the active ingredient(s).

By “‘leave-on’ composition” as used herein a topical composition is meant which after having applied to the skin, is not removed intentionally. It is preferably left on the skin for a period of at least about 15 minutes, more preferably at least about 30 minutes, even more preferably at least about 1 hour, most preferably for at least several hours, e. g. up to about 12 hours.

Of special importance as cosmetic compositions for the skin are light-protective preparations, such as sun milks, lotions, creams, oils, sunblocks or tropicals, pretanning preparations or after-sun preparations, also skin-tanning preparations (i.e. compositions for the artificial/sunless tanning and/or browning of human skin), for example self-tanning creams. Of particular interest are sun protection creams, sun protection lotions, sun protection milk and sun protection preparations in the form of a spray.

Of special importance as cosmetic compositions for the hair are preparations for hair treatment, especially hair-washing preparations in the form of shampoos, hair conditioners, hair-care preparations, e.g. pretreatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

A shampoo may, for example, have the following composition: from 0.01 to 5% by weight of a polymer according to the invention, 12.0% by weight of sodium laureth-2-sulfate, 4.0% by weight of cocamidopropyl betaine, 3.0% by weight of sodium chloride, and water ad 100%.

The fatty substances can be an oil or a wax, or mixture thereof. By the term “oil” is intended a compound which is liquid at ambient temperature. By the term “wax” is intended a compound which is solid or substantially solid at ambient temperature and for which the melting point is generally greater than 35.degree. C.

Exemplary oils are mineral oils (liquid paraffin); vegetable oils (sweet almond, macadamia, blackcurrant seed or jojoba oil); synthetic oils, such as perhydrosqualene, fatty alcohols, acids or esters (such as the C.sub.12 -C.sub.15 alkyl benzoate marketed under the trademark “Finsolv TN” by Finetex, octyl palmitate, isopropyl lanolate or triglycerides, including those of capric/caprylic acids), or oxyethylenated or oxypropylenated fatty esters and ethers; silicone oils (cyclomethicone, polydimethylsiloxanes or PDMS); fluorinated oils; polyalkylenes and their mixtures.

Exemplary waxy compounds are paraffin wax, carnauba wax, beeswax or hydrogenated castor oil.

And exemplary organic solvents include the lower alcohols and polyols having at most 8 carbon atoms.

The thickeners are advantageously selected, in particular, from among the crosslinked polyacrylic acids or modified or unmodified guar gums and celluloses, such as hydroxypropylated guar gum, methylhydroxyethylcellulose and hydroxypropylmethylcellulose.

Where convenient, other conventional UV-A and/or UV-B and/or broad spectrum UV-filter substances may be added into the topical compositions of the invention. The combination of UV-filter substances may show a synergistic effect. These additional UV-filter substances are advantageously selected from among the compounds listed below without being limited thereto:

Examples of UV-B or broad spectrum filter substances, i.e. substances having absorption maximums between about 290 nm and 340 nm may be organic or inorganic compounds.

Organic UV-B or broadband screening agents are e.g. acrylates such as 2-ethylhexyl 2-cyano-3,3-diphenylacrylate (octocrylene, PARSOL® 340), ethyl 2-cyano-3,3-diphenylacrylate and the like; camphor derivatives such as 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, therephthalidene dicamphor sulfonic acid and the like; Cinnamate derivatives such as ethylhexyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes; p-aminobenzoic acid derivatives, such as p-aminobenzoic acid, 2-ethylhexyl p-dimethylaminobenzoate, N-oxypropylenated ethyl p-aminobenzoate, glyceryl p-aminobenzoate; benzophenones such as benzophenone-3, benzophenone-4, 2,2′,4,4′-tetrahydroxy-benzophenone, 2,2′-dihydroxy-4,4′-dimethoxybenzophenone and the like; esters of benzalmalonic acid such as di-(2-ethylhexyl) 4-methoxybenzalmalonate; esters of 2-(4-ethoxy-anilinomethylene)propandioic acid such as 2-(4-ethoxy anilinomethylene) propandioic acid diethyl ester as described in the European Patent Publication EP 0895 776; organosiloxane compounds containing benzmalonate groups as described in the European Patent Publications EP 0358584 B1, EP 0538431 B1 and EP 0709080 A1 such as PARSOL® SLX; drometrizole trisiloxane (Mexoryl XL); imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts, diethanolamine salts and the like; salicylate derivatives such as isopropylbenzyl salicylate, benzyl salicylate, butyl salicylate, ethylhexyl salicylate (PARSOL® EHS, Neo Heliopan OS), isooctyl salicylate or homomenthyl salicylate (homosalate, PARSOL® HMS, Neo Heliopan HMS) and the like; triazine derivatives such as ethylhexyl triazone (Uvinul T-150), diethylhexyl butamido triazone (Uvasorb HEB) and the like.

Encapsulated UV-filters such as encapsulated ethylhexyl methoxycinnamate (Eusolex UV-pearls) or microcapsules loaded with UV-filters as e.g. dislosed in EP 1471995 and the like; Examples of broad spectrum or UV A screening agents i.e. substances having absorption maximums between about 320 nm and 400 nm may be organic or inorganic compounds e.g. dibenzoylmethane derivatives such as 4-tert.-butyl-4′-methoxydibenzoyl-methane (PARSOL® 1789), dimethoxydibenzoylmethane, isopropyldibenzoylmethane and the like; benzotriazole derivatives such as 2,2′-methylene-bis-(6-(2H-benzotriazole-2-yl)-4-(1,1,3,3,-tetramethylbutyl)-phenol (Tinosorb M) and the like; bis-ethylhexyloxyphenol methoxyphenyl triazine (Tinosorb S) and the like; phenylene-1,4-bis-benzimidazolsulfonic acids or salts such as 2,2-(1,4-phenylene)bis-(1H-benzimidazol-4,6-disulfonic acid) (Neoheliopan AP); amino substituted hydroxybenzophenones such as 2-(4-Diethylamino-2-hydroxy-benzoyl)-benzoic acid hexylester (Uvinul A plus) as described in the European Patent Publication EP 1046391; Ionic UV-A filters as described in the International Patent Publication WO2005080341 A1; Pigments such as microparticulated ZnO and the like. The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art. As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g. 3,3-Diphenylacrylate derivatives as described in the European Patent Publications EP 0 514 491 B1 and EP 0 780 119 A1; Benzylidene camphor derivatives as described in the U.S. Pat. No. 5,605,680; Organosiloxanes containing benzmalonate groups as described in the European Patent Publications EP 0358584 B1, EP 0538431 B1 and EP 0709080 A1.

The UV-filter substances are generally present in the compositions according to the invention in proportions ranging from 0.1 to 20 wt.-%, preferably ranging from 0.2 to 15 wt.-%, most preferably ranging from 0.5 to 10 wt.-% with respect to the total weigh of the composition.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The compositions according to the invention are preferably formulated an oil-in-water or water-in-oil emulsion.

The cosmetic and/or dermatological compositions according to the invention have a pH in the range of 3-10, preferably in the range of pH of 4-8, most preferred in the range of pH 4-6.

The following examples are provided to further illustrate the compounds and compositions of the present invention. These examples are illustrative only and are not intended to limit the scope of the invention in any way.

### EXAMPLE 1

1. Preparation of the hyperbranched dendritic polyether Poly-(methyl-3-oxetanmethanol)

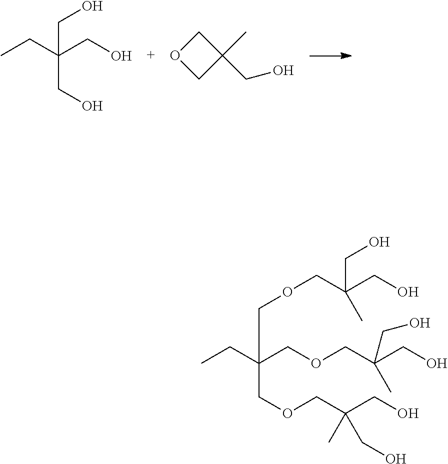

A mixture of 1,1,1-trimethylolpropane (5.4g, 40 mmol) and a 3.7 M potassium methylate solution in methanol (3.5 mL) was heated to 60° C. and methanol was removed under vacuum. The temperature was raised to 95° C. and residual methanol was removed under high vacuum. 3-methyl-3-oxetanmethanol (36.8 g, 360 mmol) was added drop wise (0.05 mL/min). The reaction mixture was stirred at 150° C. for 24h and at 185° C. for another 24 h. The reaction process was continuously followed by GPC analysis (RI detection). After cooling to room temperature the resulting polymer was solved in methanol (200 mL) and neutralized by ion exchange chromatography (Amberlite CG-120, H+ form). Evaporation under vacuum afforded 33 g poly-(methyl-3-oxetanmethanol) as highly viscous oil. Characterization of the product by GPC and ¹H NMR revealed the complete consumption of monomers.

2. Attachment of p-dimethylamino benzoyl-groups to Poly-(methyl-3-oxetanmethanol).

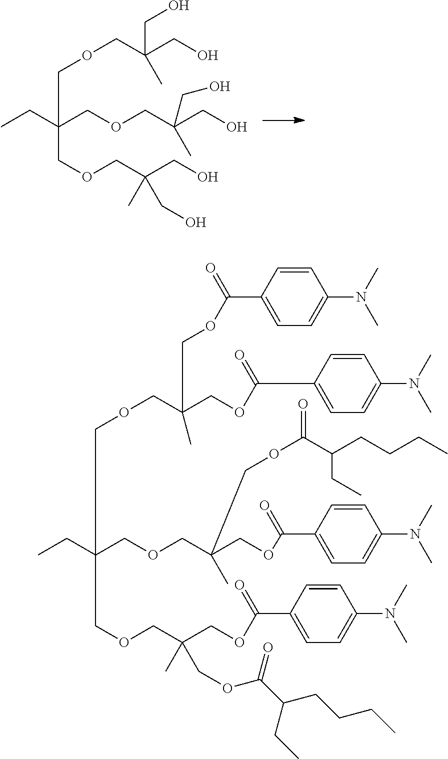

To a solution of poly-(methyl-3-oxetanmethanol) (2.0 g, 22.8 mmol OH) in pyridine (30 mL) was added p-dimethylamino benzoic acid chloride (2.9 g, 16.0 mmol, 70% loading) and the reaction mixture was stirred at 115° C. for 12 h. The reaction mixture was cooled to 80° C. and 2-ethyl hexanoic acid chloride (2.2 g, 13.7 mmol) was added. The reaction mixture was stirred at 115° C. for 4h and evaporated to dryness. The residue was dissolved in ethyl acetate (100 mL) and extracted with an aqueous potassium carbonate solution (10%, 2×75 mL), with an aqueous citric acid solution (5%, 75 mL), and brine (50 mL). The organic layer was dried over sodium sulfate, filtered, and evaporated to dryness. After removal of residual solvents at 80° C. under high vacuum for 12 h 5.1 g product was obtained as a glassy solid polymeric UVB filter, which could be ground to powder.

The polymeric filter showed an E_{1/1}-value of 812 in THF at 308 nm. The solubility in Finsolv TN (C₁₂-₁₅ alkyl benzoate) was determined to be at least 25% (w/w). The Mn has been determined by GPC using polystyrene standards to be 1700 g/mol.

### COMPARATIVE EXAMPLE

Preparation of a Polymeric UV-Filter by Attaching p-dimethylamino benzoic acid to Polyglycerol.

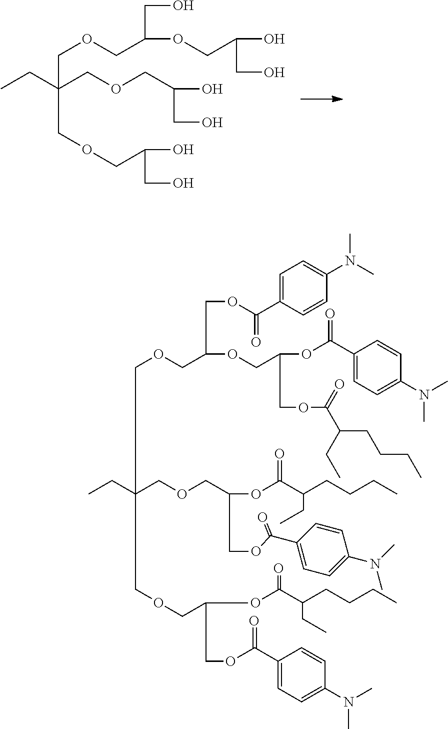

Polyglycerol was prepared according to Sunder, A.; Mülhaupt, R.; Frey, H. Macromolecules, 2000, 33, 309-314.

To a solution of polyglycerol (2.3 g, 33.8 mmol OH) in pyridine (30 mL) was added p-dimethylamino benzoic acid chloride (4.3 g, 23.7 mmol, 70% loading) and the reaction mixture was stirred at 115° C. for 12 h. The reaction mixture was cooled to 80° C. and 2-ethyl hexanoic acid chloride (3.3 g, 20.3 mmol) was added. The reaction mixture was stirred at 115° C. for 6 h. After cooling to room temperature water was added (50 mL) and the mixture was stirred at 25° C. for 12 h and evaporated to dryness. The residue was dissolved in toluene (100 mL) and extracted with an aqueous potassium carbonate solution (10%, 2×50 mL), with an aqueous citric acid solution (5%, 50 mL), and brine (20 mL). The organic layer was dried over sodium sulfate, filtered, and evaporated to dryness. After removal of residual solvents at 80° C. under high vacuum for 12h 6.5 g product was obtained as a highly viscous polymeric UVB filter.

The polymeric filter showed an E_{1/1}-value of 805 in THF at 310 nm. The product showed a solubility of less than 5wt.-% in Finsolv TN at 50° C. The Mn has been determined by GPC using polystyrene standards to be 1670 g/mol.

The solubility was measured by adding to a mixture of 50 mg of the polymeric UV filter and 150 mg of Finsolv TN in 5 minutes intervals subsequently additional 100 mg portions of Finsolv TN at 50° C. until the polymer has been completely dissolved. After each solvent addition the sample was cooled to room temperature and the solubility was checked. The solution remains clear and transparent when the polymer is completely solved. Insoluble polymers result in a cloudy liquid, which separates on standing in a polymer and a solvent phase.

### Results:

| | Example 1 | Comparative Example |
|---|---|---|
| E_{1/1} | 812 | 805 |
| Mn | 1700 g/mol | 1670 g/mol |
| Solubility | 25% | <5wt.-%* |

*After a total of 950 mg Finsolv TN was reached the experiment was skipped

As can be seen from the table, the compound according to the invention shows a higher solubility at equal chromophore loading and molecular weight compared to the respective glycidol based UV-filter substance.

### EXAMPLE 2

The in vitro SPF (Sun Protection Factor) of the following topical composition was determined according to the COLIPA protocol as described in the Guideline “Method for the in vitro determination of UVA protection provided by sunscreen products” to give an in vitro SPF of 8.

| Compound name | INCI name | % |
|---|---|---|
| Estol 3650 | Glyceryl Myristate | 2.0 |
| Butylated Hydroxytoluene | BHT | 0.05 |
| Brij 72 | Steareth-2 | 2.0 |
| Brij 721 | Steareth-21 | 2.0 |
| Lanette O | Cetearyl Alcohol | 2.0 |
| Finsolv TN | C12-15 Alkyl Benzoate | 18.0 |
| Phenonip | Phenoxyethanol & Methylparabene | 0.8 |
| PABA-Polyoxetane | Example 1 | 8.0 |
| EDTA BD | Disodium EDTA | 0.1 |
| Glycerin | Glycerin | 4.0 |
| Potassium Hydroxide 10% | Potassium Hydroxide | 0.12 |
| Water | Aqua | 59.93 |
| Sepigel 305 | Polyacrylamide & C13-14 | 1.0 |
| | Isoparabene | |

1. A polymer comprising a hyperbranched dendritic polyether backbone covalently bonded via an oxygen bridge to at least one UV absorbing chromophore characterized in that the hyperbranched dendritic polyether backbone is obtainable by a ring-opening polymerization of at least one oxetane.

2. A polymer as in claim 1, characterized in that the oxetane is a compound of the general formula (I) 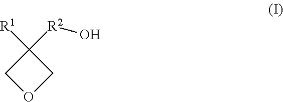
wherein
R¹ represents H, a C₁ to C₄ alkyl group, an aryl group or an arylalkyl group and
R² represents a C₁ to C₄ alkylene group.

3. A polymer as in claim 1, wherein the oxetane is 3-methyl-3-hydroxymethyl oxetane.

4. A polymer as in claim 1, wherein a polyol starter unit is present.

5. A polymer as in claim 4, wherein the polyol starter unit is trimethylolpropane.

6. A polymer as in claim 1, wherein the UV absorbing chromophore is a p-aminobenzoyl-, amino substituted hydroxybenzophenone-, cinnamoyl- and/or a benzalmalonate group.

7. A polymer as in claim 1 wherein the UV absorbing chromophore is a p-dimethylaminobenzoyl-group.

8. A polymer as in claim 1, wherein 70-90% of the terminal hydroxyl groups of the hyperbranched dendritic polyether backbone are linked to a UV absorbing chromophore and the residual terminal hydroxyl groups are linked to a capping group.

9. A polymer as in claim 8, wherein the capping group is a 2-ethyl hexanoyl-group.

10. A topical composition comprising a polymer as in claim 1 and a cosmetically acceptable carrier.

11. Use of a polymer as in claim 1 as UV-filter substance.
